(19) United States
(12) Patent Application Publication
(10) Pub. No.: US 2008/0064625 A1
(43) Pub. Date: Mar. 13, 2008

(54) 4-Phenylpentan-2-ol as a fragrance and flavouring

(75) Inventor: Bernd Holscher, Halle (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & Co. KG, Holzminden (DE)

(21) Appl. No.: 11/852,577

(22) Filed: Sep. 10, 2007

(30) Foreign Application Priority Data
Sep. 11, 2006 (DE) 10 2006 043 226.6

(51) Int. Cl. A61K 8/34; A23L 1/221; A61Q 13/00; C07C 33/18

(52) U.S. Cl. 512/20; 426/650; 568/715

(57) ABSTRACT

The use of 4-phenylpentan-2-ol (I)

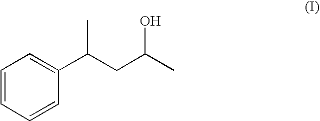

as a fragrance or flavouring is described.

### FIELD OF THE INVENTION

The present invention relates to the use of 4-phenylpentan-2-ol as a fragrance and flavouring. The invention also relates to perfumed or flavoured articles containing this compound and to corresponding processes for imparting, modifying and/or intensifying certain odour and/or flavour notes. In addition, processes for the preparation of 4-phenylpentan-2-ol are described.

### BACKGROUND OF THE INVENTION

Despite a large number of already existing fragrances, a general need for new fragrances continues to exist in the perfume industry. For example, there is a need for fragrances with interesting green perfume notes which are capable of producing (in fragrance compositions) other interesting odour notes in addition to a green perfume note, such as for example fruity notes, and of expanding the perfumer's options with their novel or original perfume properties. In particular, there is interest in fragrances with green and fruity perfume notes which are capable of entering into a harmonious combination with floral-scented fragrances.

A superimposing of the different odour aspects and notes should preferably take place, thereby creating a complex overall odour impression.

For the creation of novel modern compositions, there is a constant need for fragrances with particular odour properties, which are suitable for use as a base for the composition of novel modern perfumes with a complex odour character. Preferred fragrances being sought should exhibit other notes and aspects apart from a green, fruity perfume note, which give them odour character and complexity.

The search for suitable fragrances which led to the present invention was made more difficult by the following facts:
The mechanisms of odour perception are not sufficiently well known.
The associations between the specific odour perception on the one hand and the chemical structure of the related fragrance on the other hand have not been adequately researched.
Even small changes in the structural composition of a known Fragrance often bring about marked changes in the sensory properties and negatively affect compatibility for the human organism

The success of the search for suitable fragrances is therefore heavily dependent on the intuition of the researcher.

### SUMMARY OF THE INVENTION

The object on which this invention is based therefore consisted substantially in finding fragrances with green, fruity perfume notes, which are preferably coupled with other interesting and original odour properties, as a result of which the fragrances sought make novel and original fragrance compositions possible with particular odour notes and aspects. In particular, fragrances with green, fruity perfume notes were to be found which are particularly suitable for combining with fragrances having a woody and/or floral perfume note.

At the same time, the fragrances achieving this main object should preferably also have additional positive secondary properties beyond their primary, i.e. odour, properties, such as e.g. higher stability under certain application conditions, higher diffusivity, better tenacity, high substantivity, a noticeable booster effect or a marked blooming, so that sensorially noticeable effects or else better dermatological and toxicological properties as compared to similar fragrances can be achieved.

According to the invention, the primary object set is achieved by the use of 4-phenylpentan-2-ol of formula (I)

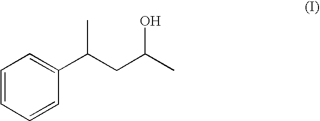

as a fragrance or flavouring.

The 4-phenylpentan-2-ol of (I) possesses two chiral centres. The compound of formula (I) can be syn- or anti-configured or can be present as an (R,R) configured enantiomer, (R,S) configured enantiomer, (S,R) configured enantiomer, (S,S) configured or any mixture of enantiomers, particularly as a racemate, or as any mixture of the corresponding diastereomers.

The object set is additionally achieved by the use of 4-phenylpentan-2-ol of (I) as a fragrance or flavouring with green, fruity notes.

Other aspects of the invention can be taken from the attached claims and the following description; these aspects relate particularly to new perfumed or flavoured articles and to corresponding processes.

### DETAILED DESCRIPTION OF THE INVENTION

The odour properties of the compound 4-phenylpentan-2-ol of (I) to be used according to the invention are described as follows by perfumers: very strongly radiant, interesting green, fruity fragrance note in the direction of grapefruit and rhubarb, very natural odour impression.

In particular, the compound of formula (I) can be used to impart, to modify and/or to intensify a green, fruity odour and/or flavour note.

In J. Chem. Soc., Chem. Commun. (1982), 348, the synthesis of (R,S)-4-phenylpentan-2-ol by the hydrogenation of chiral homoallyl alcohols is described.

Eur. J. Org. Chem. 2002, 16, 2472-2757, J. Org. Chem. 2000, 65, 4281-4288, Chem. Commun. 1998, 19, 2127-2128 and Tetrahedron Lett. 1998, 39, 8829-8832 deal with stereoselective radical aryl migration, the preparation of 4-phenylpentan-2-ol of formula (I) also being investigated in detail.

No sensory description of 4-phenylpentan-2-ol of formula (I) has been provided until now.

In the Journal f. prakt. Chemie vol. 319, part 4, 1977, 601, the catalytic cleavage of substituted 1,3-dioxanes is described, but the compound to be used according to the invention, 4-phenylpentan-2-ol, is not mentioned in this article.

The odour descriptions of phenylpentanols known from in-house investigations and from the literature (S. Arctander, Perfume and Flavor Materials, vol. I and II, Montclair, N.J. 1969, self-published) can be summarised as follows:
5-Phenylpentan-1-ol: sweet-herbal, citrus note, verbena note
5-Phenylpentan-3-ol: balsamic, floral, earthy-green-spicy
1-Phenylpentan-2-ol: slightly green, sweet, damp-earthy

There is also a certain structural similarity of the compound of formula (I) to be used according to the invention to the tertiary alcohol 4-phenyl-2-methyl-2-butanol (α,α-dimethylphenylethylcarbinol) known as a fragrance, but this has a dry, rose-floral, lily-like odour.

Other phenyl alkanols having side chains with more or fewer C atoms than the compound of formula (I) to be used according to the invention also display odour characteristics different from this (cf. Common Fragrance and Flavor Materials, 4th Edition, Wiley-VCH, Weinheim 2001):

Dihydrocinnamyl alcohol (3-phenylpropanol) possesses a fruity cinnamon odour.

2-Methyl-5-phenylpentanol (commercially available under the name Rosaphen) has a fragrance of rose blossoms with a slightly waxy note.

3-Methyl-5-phenylpentanol (commercially available under the name Phenoxanol) is known for its long-lasting, diffusive, fresh, floral rose odour.

According to the prior art described, the area of the phenylpentanols is well investigated and it is therefore particularly unexpected that another valuable fragrance could be found in this area. The compound of formula (I) has completely independent olfactory properties, which clearly stand out from the known fragrances of similar structure. The suitability of the compound of formula (I) to be used according to the invention as a fragrance or flavouring for use in the fragrance and flavour industry was not known until now and is surprising.

It is particularly surprising that the 4-phenylpentan-2-ol to be used according to the invention has an expressive rhubarb note with additional, in some cases very complex and multi-faceted, aspects in addition to the green-fruity aspects since, owing to the presence of these other additional aspects, the compound to be used according to the invention differs very markedly from structurally comparable and known substances (see above) and imparts a very complex and diverse odour and taste impression which can otherwise only be achieved by complex mixtures of several components (such as e.g. by essential oils or herb and/or spice mixes).

In mixtures with other fragrances, the 4-phenylpentan-2-ol to be used according to the invention is able, even in small doses, to enhance the intensity of a fragrance mixture and to round out and harmonise the overall effect of the fragrance mixture as well as to give more radiance and naturalness to the mixture.

Within the framework of the use according to the invention of 4-phenylpentan-2-ol as a fragrance or flavouring (particularly with a green fruity note), the present invention also relates to its use for providing (a) hair, (b) skin or (c) textile fibres with a perfume, particularly a green, fruity perfume (see above for other odour or flavour notes). The present invention also relates to corresponding processes and (preferably surfactant-containing) mixtures. According to a related aspect, the present invention also concerns the use of 4-phenylpentan-2-ol as a means for increasing the substantivity and/or retention of a fragrance mixture and/or as a fixative and/or as a means for increasing the odour of other fragrances perceived via a surfactant-containing aqueous solution.

Connected with the preferred use for modifying and/or intensifying an odour and/or flavour note, there is also the finding that the 4-phenylpentan-2-ol to be used according to the invention can act outstandingly well as a so-called booster (intensifier; enhancer).

The 4-phenylpentan-2-ol to be used according to the invention possesses additional positive secondary properties beyond its primary, i.e. odour, properties, such as e.g. high stability under certain application conditions, high diffusivity, good tenacity and high substantivity.

4-Phenylpentan-2-ol of (I) can particularly be used to give radiance, rounding and/or harmony to a fragrance or flavouring composition and/or to intensify existing odour and/or flavour notes.

The present invention also relates to perfumed or flavoured articles comprising

(a) the use of 4-phenylpentan-2-ol of (I), preferably in a quantity sufficient to impart, to modify and/or to intensify an odour or flavour note of the green, fruity type, particularly in the direction of grapefruit and rhubarb, and

(b) one, two, three or more other fragrances or flavourings, the other fragrance(s) or flavouring(s) preferably imparting a woody and/or floral odour and/or flavour.

The preferred configurations of the invention described above and below with regard to the uses according to the invention also apply correspondingly to perfumed or flavoured articles according to the invention

4-Phenylpentan-2-ol is usually employed within the framework of the use according to the present invention in sensorially active quantities. The 4-phenylpentan-2-ol to be used according to the invention is frequently to be mixed or combined with other fragrances or flavourings. In this case, the weight ratio of the total quantity of 4-phenylpentan-2-ol to the total quantity of other fragrances or flavourings is preferably in the range of 1:1000 to 1:0.5, preferably 1:100 to 1:0.5.

A perfumed or flavoured article according to the invention is preferably a fragrance or flavouring composition. By combining 4-phenylpentan-2-ol with one, two, three or more other fragrances or flavourings (preferably having a woody and/or floral odour or flavour), new fragrance or flavouring compositions can be formed. In this way, particularly interesting and natural new and original perfume notes can be created. Fragrances (as component (b)) which are advantageously suitable for combining with 4-phenylpentan-2-ol can be found e.g. in S. Arctander, Perfume and Flavor Materials, vol. I and II, Montclair, N.J. 1969, self-published, or K. Bauer et al., Common Fragrance and Flavor Materials, 4th Edition, Wiley-VCH, Weinheim 2001. The following are mentioned in detail:

(i) extracts of natural raw materials, such as essential oils, concretes, absolutes, resins, resinoids, balsams, tinctures such as e.g.

ambergris tincture; amyris oil; angelica seed oil; angelica root oil; aniseed oil; valerian oil; basil oil; tree moss absolute; bay oil; armoise oil; benzoin resin; bergamot oil; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oakmoss absolute; elemi oil; tarragon oil; eucalyptus citriodora oil; eucalyptus oil; fennel oil; fir needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemon-grass oil; lovage oil; lime oil distilled; lime oil expressed; linaloe oil; Litsea cubeba oil; laurel leaf oil; mace oil; marjoram oil; mandarin oil; massoi bark oil; mimosa absolute; ambrette seed oil; musk tincture; clary sage oil; nutmeg oil; myrrh absolute; myrrh oil; myrtle oil; clove leaf oil; clove bud oil; neroli oil; olibanum absolute; olibanum oil; opopanax oil; orange flower absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil: spike-lavender oil; star anise oil; styrax oil; tagetes oil; fir needle oil; tea tree oil; turpentine oil; thyme oil; Tolu balsam; tonka bean absolute; tuberose absolute; vanilla extract; violet leaf absolute; verbena oil; vetiver oil; juniper berry oil; wine lees oil; wormwood oil; wintergreen oil; ylang-ylang oil; hyssop oil; civet absolute; cinnamon leaf oil; cinnamon bark oil and fractions thereof or ingredients isolated therefrom;

(ii) individual fragrances from the group comprising the hydrocarbons, such as e.g. 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;

the aliphatic alcohols, such as e.g. hexanol; octanol; 3-octanol; 2,6-dimethylheptanol; 2-methyl-2-heptanol, 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol; the aliphatic aldehydes and their acetals, such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanaldiethylacetal; 1,1-dimethoxy-2,2,5-trimethyl-4-hexene; citronellyl oxyacetaldehyde; 1-(1-methoxypropoxy)-(E/Z)-3-hexene;

the aliphatic ketones and oximes thereof, such as e.g. 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;

the aliphatic sulfur-containing compounds, such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

the aliphatic nitriles, such as e.g. 2-nonenenitrile; 2-undecenenitrile; 2-tridecenenitrile; 3,12-tridecadienenitrile; 3,7-dimethyl-2,6-octadienenitrile; 3,7-dimethyl-6-octenenitrile;

the esters of aliphatic carboxylic acids, such as e.g. (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl-2-methyl pentanoate; ethyl hexanoate; allyl hexanoate; ethyl heptanoate; allyl heptanoate; ethyl octanoate; ethyl-(E,Z)-2,4-decadienoate; methyl-2-octinate; methyl-2-noninate; allyl-2-isoamyl oxyacetate; methyl-3,7-dimethyl-2,6-octadienoate, 4-methyl-2-pentyl crotonate;

the acyclic terpene alcohols, such as e.g. citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol 2,6-dimethyl-2,5,7-octatrien-1-ol; as well as formates, acetates, propionates, isobutyrates, butyrates, isovalerates, pentanoates, hexanoates, crotonates, tiglinates and 3-methyl-2-butenoates thereof;

the acyclic terpene aldehydes and ketones, such as e.g. geranial; neral; citronellal; 7-hydroxy-3,7-dimethyloctanal; 7-methoxy-3,7-dimethyloctanal; 2,6,10-trimethyl-9-undecenal; geranylacetone; as well as the dimethyl- and diethylacetals of geranial, neral and 7-hydroxy-3,7-dimethyloctanal;

the cyclic terpene alcohols, such as e.g. menthol; isopulegol; alpha-terpineol; terpinen-4-ol; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates and 3-methyl-2-butenoates thereof;

the cyclic terpene aldehydes and ketones, such as e.g. menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methyl cedryl ketone);

the cyclic alcohols, such as e.g. 4-tert.-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

the cycloaliphatic alcohols, such as e.g. alpha,3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

the cyclic and cycloaliphatic ethers, such as e.g. cineole; methyl cedryl ether; cyclododecyl methyl ether; 1,1-dimethoxycyclododecane; (ethoxymethoxy)cyclododecane; alpha-cedrene epoxide; 3a,6,6,9a-tetramethyldodecahydronaphtho[2,1-b]furan; 3a-ethyl-6,6,9a-trimethyidodecahydro-naphtho[2,1-b]furan; 1,5,9-trimethyl-1 3-oxabicyclo-[10.1.0]-trideca-4,8-diene; rose oxide; 2-(2,4-dimethyl-3-cyclohexen-1-yl)-5-methyl-5-(1-methylpropyl)-1,3-dioxane; 2,4,6-trimethyl-4-phenyl-1,3-dioxane (Vertacetal®, preferred combination, see below)

the cyclic and macrocyclic ketones, such as e.g. 4-tert.-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert.-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;

the cycloaliphatic aldehydes, such as e.g. 2,4-dimethyl-3-cyclohexene carbaldehyde; 2-methyl-4-(2,2,6-trimethylcyclohexen-1-yl)-2-butenal; 4-(4-hydroxy-4-methylpentyl)-3-cyclohexene carbaldehyde; 4-(4-methyl-3-penten-1-yl)-3-cyclohexene carbaldehyde;

the cycloaliphatic ketones, such as e.g. 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert.-butyl-(2,4-dimethyl-3-cyclohexen-1-yl) ketone;

the esters of cyclic alcohols, such as e.g. 2-tert.-butylcyclohexyl acetate; 4-tert.-butylcyclohexyl acetate; 2-tert.-pentylcyclohexyl acetate; 4-tert.-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or 6-indenyl acetate; 4,7-methano-3a,4,5,6,7,7a-hexahydro-5- or 6-indenyl propionate; 4,7-methano-3a,4,5,6,7,7a-hexahydro-5- or 6-indenyl isobutyrate; 4,7-methanooctahydro-5 or 6-indenyl acetate;

the esters of cycloaliphatic alcohols, such as e.g. 1-cyclohexylethyl crotonate;

the esters of cycloaliphatic carboxylic acids, such as e.g. allyl 3-cyclohexylpropionate; allyl cyclohexyl oxyacetate; cis- and trans-methyl dihydrojasmonate; cis- and transmethyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexene-carboxylate; ethyl 2-methyl-1,3-dioxolane-2-acetate;

the araliphatic alcohols, such as e.g. benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenyl-propanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

the esters of araliphatic alcohols and aliphatic carboxylic acids, such as e.g. benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;

the araliphatic ethers, such as e.g. 2-phenylethyl methyl ether; 2-phenylethyl isoamyl ether; 2-phenylethyl-1-ethoxyethyl ether; phenylacetaldehyde dimethyl acetal; phenylacetaldehyde diethyl acetal; hydratropaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

the aromatic and araliphatic aldehydes, such as e.g. benzaldehyde; phenylacetaldehyde; 3-phenylpropanal; hydratropaldehyde; 4-methylbenzaldehyde; 4-methylphenylacetaldehyde; 3-(4-ethyl phenyl )-2,2-dimethylpropanal; 2-methyl-3-(4-isopropylphenyl)propanal; 2-methyl-3-(4-tert.-butylphenyl)propanal; 2-methyl-3-(4-isobutylphenyl)propanal; 3-(4-tert.-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

the aromatic and araliphatic ketones, such as e.g. acetophenone; 4-methylacetophenone; 4-methoxyacetophenone; 4-tert.-butyl-2,6-dimethyl-acetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tert.-butyl-1,1-dimethyl-4-indanyl methyl ketone; 1-[2,3-dihydro-1,1,2,6-tetramethyl-3-(1-methyl-ethyl)-1H-5-indenyl]ethanone; 5′,6′,7′,8′-tetrahydro-3′,5′, 5′,6′,8′,8′-hexamethyl-2-acetonaphthone;

the aromatic and araliphatic carboxylic acids and esters thereof, such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

the nitrogen-containing aromatic compounds, such as e.g. 2,4,6-trinitro-1,3-dimethyl-5-tert.-butylbenzene; 3,5-dinitro-2,6-dimethyl-4-tert.-butylacetophenone; cinnamonitrile; 3-methyl-5-phenyl-2-pentenenitrile; 3-methyl-5-phenylpentanenitrile; methyl anthranilate; methyl-N-methylanthranilate; Schiff's bases of methyl anthranilate with 7-hydroxy-3,7-dimethyloctanal, 2-methyl-3-(4-tert.-butylphenyl)propanal or 2,4-dimethyl-3-cyclohexene carbaldehyde; 6-isopropylquinoline; 6-isobutylquinoline; 6-sec.-butylquinoline; 2-(3-phenylpropyl)pyridine; indole; skatole; 2-methoxy-3-isopropyl-pyrazine; 2-isobutyl-3-methoxypyrazine;

the phenols, phenyl ethers and phenyl esters, such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;

the heterocyclic compounds, such as e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

the lactones, such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene-1,12-dodecanedioate; ethyl-ene-1,13-tridecanedioate; coumarin; 2,3-dihydrocoumarin; octahydrocoumarin.

A fragrance or flavouring composition according to the present invention can be prepared for example by mixing 4-phenylpentan-2-ol as component (a) with one or more other fragrances or flavourings (as component (b)). Component (a) is typically used in a quantity sufficient to impart, to modify and/or to intensify an odour or flavour note of the green, fruity type in the finished composition.

A fragrance or flavouring composition according to the invention preferably contains a total quantity of 4-phenylpentan-2-ol (component (a)) in the range of 0.01 to 50 wt. %, preferably 0.1 to 25 wt. % and particularly preferably 0.5 to 20 wt. %, based on the total quantity of the fragrance or flavouring composition.

Where the 4-phenylpentan-2-ol to be used according to the invention is mainly employed to give more radiance, rounding and/or harmony to a fragrance or flavouring composition and/or to intensify certain notes, its total quantity is preferably comparatively low and particularly preferably in the range of 0.01 to 5 wt. %, preferably in the range of 0.1 to 2 wt. %, based on the total quantity of the fragrance or Ilavouring composition. Where a comparatively low concentration within the preferred concentration ranges is selected, in some cases the inherent odour or flavour notes mentioned above are not yet imparted, depending on the other components of the composition in question.

The preparation of the 4-phenylpentan-2-ol to be used according to the invention can take place by means of reactions and processes that are known per se.

For example, 4-phenylpentan-2-ol of formula (I) can be prepared based on the literature (Journal f. prakt. Chemie vol. 319, part 4, 1977, 601-610) by a catalytic cleavage of substituted 1,3-dioxanes of formula (A) (see following formula diagram).

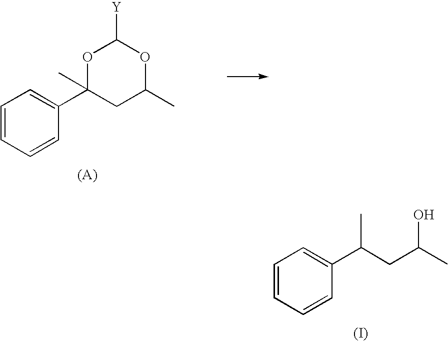

wherein Y signifies either hydrogen or an alkyl residue with 1 to 10 C atoms, Y preferably signifying methyl or ethyl.

The preferred feedstock of formula (A) is 2,4,6-trimethyl-4-phenyl-1,3-dioxane (formula (A) with Y=methyl), which is described in U.S. Pat. No. 6,114,301 and is commercially available under the name Vertacetal® or Vertacetal® Coeur (Symrise GmbH & Co. KG, Germany). This feedstock itself possesses an odour in the direction of grapefruit and rhubarb and can be combined outstandingly well with 4-phenylpentan-2-ol in order to make available novel articles such as fragrance or flavouring compositions.

The catalytic hydrogenation is performed in the presence of a hydrogenation catalyst, preferably in the range of 160-240° C. under a hydrogen pressure in the range of 20 to 50.

Suitable hydrogenation catalysts can contain for example ruthenium, rhodium, iridium, nickel, palladium or platinum. Advantageous hydrogenation catalysts contain palladium, platinum, ruthenium or rhodium, with (elemental) palladium being a particularly preferred hydrogenation catalyst.

The hydrogenation catalysts to be used according to the invention can be applied on to organic or inorganic support materials. The hydrogenation catalysts can contain a support material or mixtures of support materials. The following may be mentioned as advantageous support materials: activated carbon, carbon, aluminium oxides, metal oxides, silica gels, zeolites, clays, clay granules, amorphous aluminium silicates and other inorganic supports. A preferred support material is activated carbon.

Especially preferred catalysts are palladium on activated carbon and palladium on aluminium oxide. The product mixture containing 4-phenylpentan-2-ol can contain residues of the dioxane of formula (A) which—particularly in the case of Vertacetal—can make a positive contribution to the overall sensory impression.

4-Phenylpentan-2-ol can also be prepared starting from 3-phenylbutyraldehyde via a Grignard reaction with MeMgX (X═Cl, Br or I) as illustrated by the formula diagram below.

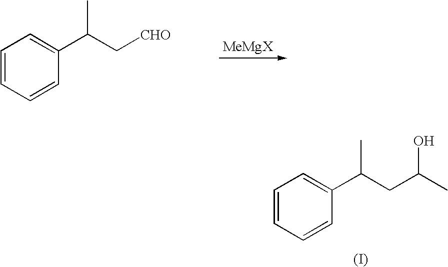

Fragrance or flavouring compositions according to the invention which contain 4-phenylpentan-2-ol of formula (I) can be used for perfuming or flavouring in liquid form, undiluted or diluted with a solvent. Suitable solvents for this purpose are e.g. ethanol, isopropanol, diethylene glycol monoethyl ether, glycerol, propylene glycol, 1,2-butylene glycol, dipropylene glycol, diethyl phthalate, triethyl citrate, isopropyl myristate, triacetin etc.

Fragrance or flavouring compositions according to the invention which contain 4-phenylpentan-2-ol of formula (I) can be adsorbed on a support which ensures both a fine distribution of the fragrances or flavourings in the product and a controlled release during application. These supports can be porous inorganic materials such as Leichtsulfat, silica gels, zeolites, gypsums, clays, clay granules, autoclaved aerated concrete etc., or organic materials such as woods, cellulose-based substances, sugars, dextrins (e.g. maltodextrin) or plastics such as PVC, polyvinyl acetates or polyurethanes. The combination of composition according to the invention and support represents an example of an article according to the invention.

Fragrance or flavouring compositions according to the invention containing 4-phenylpentan-2-ol of formula (I) can also be present in micro-encapsulated or spray-dried form or as inclusion complexes or extruded products (i.e. articles according to the invention) and can be added in this form e.g. to a product to be perfumed or flavoured.

The properties of the compositions modified in this way can optionally be further optimised by coating with suitable materials with a view to a more targeted release of perfume, with wax-like plastics such as e.g. polyvinyl alcohol preferably being used for this purpose. The resulting products again represent articles according to the invention.

The microencapsulation of the fragrance or flavouring compositions according to the invention to form articles according to the invention can take place for example by the so-called coacervation process with the aid of capsule materials, e.g. of polyurethane-like substances or soft gelatins. The spray-dried fragrance or flavouring compositions can be prepared e.g. by spray-drying an emulsion or dispersion containing the fragrance or flavouring composition, with the possible use of modified starches, proteins, dextrin and vegetable gums as supports. Inclusion complexes can be prepared e.g. by introducing dispersions of the fragrance or flavouring composition and cyclodextrins or urea derivatives into a suitable solvent, e.g. water. Extruded products can be obtained by melting the fragrance or flavouring compositions with a suitable waxy substance and by extrusion with subsequent setting, optionally in a suitable solvent, e.g. isopropanol.

4-Phenylpentan-2-ol of (I) and fragrance or flavouring compositions containing 4-phenylpentan-2-ol can be used in concentrated form, in solutions or in a modified form described above for the production of perfumed articles according to the invention, such as e.g. perfume extracts, eaux de parfum, eaux de toilette, aftershaves, eaux de cologne, pre-shave products, splash colognes and perfumed freshening wipes, and the perfuming of acidic, alkaline and neutral cleaners, such as e.g. floor cleaners, window cleaners, dishwashing detergents, bath and sanitary cleaners, scouring milk, solid and liquid WC cleaners, powder and foam carpet cleaners, fabric fresheners, ironing aids, liquid laundry detergents, powdered laundry detergents, laundry pre-treatment agents such as bleaches, soaking agents and stain removers, fabric softeners, laundry soaps, laundry tablets, disinfectants, surface disinfectants, and air fresheners in liquid or gel form or deposited on a solid carrier, aerosol sprays, waxes and polishes, such as furniture polishes, floor waxes, shoe creams, and toiletries, such as e.g. solid and liquid soaps, shower gels, shampoos, shaving soaps, shaving foams, bath oils, cosmetic emulsions of the oil-in-water, water-in-oil and water-in-oil-in-water types, such as e.g. skin creams and lotions, face creams and lotions, sunscreen creams and lotions, after-sun creams and lotions, hand creams and lotions, foot creams and lotions, depilatory creams and lotions, aftershave creams and lotions, tanning creams and lotions, hair-care products, such as e.g. hairsprays, hair gels, hair setting lotions, hair conditioners, permanent and semi-permanent hair colorants, hair-shaping compositions, such as cold waves and hair-smoothing compositions, hair tonics, hair creams and lotions, deodorants and antiperspirants, such as e.g. underarm sprays, roll-ons, deodorant sticks, deodorant creams, decorative cosmetic products such as e.g. eyeshadows, nail varnishes, foundations, lipsticks, mascara, as well as candles, lamp oils, incense sticks, insecticides, repellents and propellents.

4-Phenylpentan-2-ol of (I) can be incorporated into flavoured articles or articles to be flavoured, particularly preparations to be consumed for nutrition or pleasure or used for oral hygiene.

Preparations to be consumed for nutrition or pleasure are e.g. baked goods (e.g. bread, dry biscuits, cakes, other baked products), confectionery (e.g. chocolates, chocolate bar products, other bar products, fruit gums, boiled and soft sweets, chewing gum), alcoholic or non-alcoholic drinks (e.g. coffee, tea, wine, wine-containing drinks, beer, beer-containing drinks, liqueurs, spirits, brandies, sparkling fruit drinks, isotonic drinks, soft drinks, nectars, fruit and vegetable juices, fruit or vegetable juice preparations), instant drinks (e.g. instant cocoa drinks, instant tea drinks, instant coffee drinks), meat products (e.g. ham, fresh sausage or dry sausage preparations, spiced or marinated fresh or salt meat products), eggs or egg products (dried egg, egg white, egg yolk), cereal products (e.g. breakfast cereals, muesli bars, pre-cooked rice products), dairy products (e.g. milk drinks, dairy ice-cream, yoghurt, kefir, fresh cheese, soft cheese, hard cheese, dried powdered milk, whey, butter, buttermilk, products containing partially or completely hydrolysed milk protein), products from soy protein or other soybean fractions (e.g. soy milk and products made therefrom, preparations containing soy lecithin, fermented products such as tofu or tempe or products made therefrom, soy sauces), fruit preparations (e.g. preserves, fruit ice, fruit sauces, fruit fillings), vegetable preparations (e.g. ketchup, sauces, dried vegetables, frozen vegetables, precooked vegetables, vegetables pickled in vinegar, vegetable preserves), snack items (e.g. baked or deep-fried potato crisps or potato dough products, bread dough products, extrudates based on maize or peanuts), oil- and fat-based products or emulsions thereof (e.g. mayonnaise, remoulade, dressings, seasoning preparations), other ready meals and soups (e.g. dried soups, instant soups, pre-cooked soups), spices, seasoning mixes and particularly seasonings, which are used e.g. in the snacks sector. After incorporating the compound of formula (I) according to the invention, these preparations are preparations according to the invention (as an example of articles according to the invention).

Preparations according to the invention can be present e.g. as semi-finished products or as a seasoning mix.

Preparations according to the invention can particularly be used as semi-finished products for the production of other preparations consumed for nutrition or pleasure, particularly in spray-dried form. Preparations according to the invention can also take the form of capsules, tablets (uncoated and coated tablets, e.g. enteric coatings), dragees, granules, pellets, mixtures of solids, dispersions in liquid phases, emulsions, powders, solutions, pastes or other preparations suitable for swallowing or chewing as food supplements.

Preparations according to the invention used for oral hygiene are, in particular, oral hygiene and/or dental care agents such as toothpastes, dental gels, tooth powders, mouthwashes, chewing gums and other oral hygiene agents.

Other conventional active, basic, auxiliary and additive substances for preparations consumed for nutrition or pleasure or used in oral hygiene can be contained in quantities of 5 to 99.999999 wt. %, preferably 10 to 80 wt. %, based on the total weight of the preparation. Moreover, the preparations can contain water in a quantity of up to 99.999999 wt. %, preferably 5 to 80 wt. %, based on the total weight of the preparation.

Preparations according to the invention (as examples of articles according to the invention) containing 4-phenylpentan-2-ol are prepared according to a preferred configuration by incorporating the 4-phenylpentan-2-ol of formula (I) as a substance, as a solution (e.g. in ethanol, water or 1,2-propylene glycol) or in the form of a mixture with a solid or liquid support (e.g. maltodextrin, starch, silica gel), other flavours or flavourings and optionally other auxiliaries and/or stabilisers (e.g. natural or synthetic poly-saccharides and/or vegetable gums such as modified starches or gum arabic) into a base preparation to be consumed for nutrition or pleasure or used for oral hygiene. Preparations according to the invention which are present as a solution and/or suspension or emulsion can advantageously also be converted into a solid preparation (semi-finished product) according to the invention by spray-drying.

The spray-dried solid preparations according to the invention (as an example of articles according to the invention) are particularly suitable as semi-finished products for the production of other preparations according to the invention. Preferably 50 to 95 wt. % support materials, particularly maltodextrin and/or starch, 5 to 40% auxiliary substances, preferably natural or synthetic polysaccharides and/or vegetable gums such as modified starches or gum arabic, are contained in the spray-dried solid preparations according to the invention.

According to another preferred embodiment, to produce preparations according to the invention the 4-phenylpentan-2-ol and optionally other components of the preparation according to the invention are first incorporated into emulsions, liposomes, e.g. starting from phosphatidylcholine, microspheres, nanospheres or into capsules, granules or extrudates of a matrix suitable for foodstuffs and products consumed for pleasure, e.g. of starch, starch derivatives (e.g. modified starch), cellulose or cellulose derivatives (e.g. hydroxypropylcellulose), other polysaccharides (e.g. dextrin, alginate, curdlan, carrageenan, chitin, chitosan, pullulan), natural fats, natural waxes (e.g. beeswax, carnauba wax), of proteins, e.g. gelatin, or other natural products (e.g. shellac). Depending on the matrix, the products can be obtained by spray drying, spray granulation, melt granulation, coacervation, coagulation, extrusion, melt extrusion, emulsion processes, coating or other suitable encapsulation processes and optionally a suitable combination of the aforementioned processes. In another preferred production process for a preparation according to the invention, the 4-phenylpentan-2-ol is first complexed with one or more complexing agents, e.g. with cyclodextrins or cyclodextrin derivatives, preferably α or β-cyclodextrin, and used in this complexed form.

A preparation according to the invention in which the matrix is selected such that the release of the 4-phenylpentan-2-ol of formula (I) from the matrix is delayed, so that a long-lasting effect is achieved, is particularly preferred. In this respect, a fat, wax, polysaccharide or protein matrix is particularly preferred.

Conventional basic, auxiliary and additive substances for foodstuffs or products consumed for pleasure can be used as other components of preparations consumed for nutrition or pleasure according to the invention, e.g. water, mixtures of fresh or processed vegetable or animal basic materials or raw materials (e.g. raw, roasted, dried, fermented, smoked and/or boiled meat, bone, cartilage, fish, vegetables, fruits, herbs, nuts, vegetable or fruit juices or pastes or mixtures thereof), digestible or indigestible carbohydrates (e.g. sucrose, maltose, fructose, glucose, dextrins, amylose, amylopectin, inulin, xylans, cellulose, tagatose), sugar alcohols (e.g. sorbitol, erythritol), natural or hydrogenated fats (e.g. tallow, lard, palm kernel fat, coconut fat, hydrogenated vegetable fat), oils (e.g. sunflower oil, peanut oil, corn oil, olive oil, fish oil, soy-bean oil, sesame seed oil), fatty acids or their salts (e.g. potassium stearate), proteinogenic or non-proteinogenic amino acids and related compounds (e.g. γ-aminobutyric acid, taurine), peptides (e.g. glutathione), native or processed proteins (e.g. gelatin), enzymes (e.g. peptidases), nucleic acids, nucleotides, flavour-masking agents for unpleasant flavour impressions, other flavour modulators for other, generally not unpleasant, flavour impressions, other flavour-modulating substances (e.g. inositol phosphate, nucleotides such as guanosine monophosphate, adenosine monophosphate or other substances such as monosodium glutamate or 2-phenoxypropionic acid), emulsifiers (e.g. lecithins, diacylglycerols, gum arabic), stabilisers (e.g. carrageenan, alginate), preservatives (e.g. benzoic acid, sorbic acid), antioxidants (e.g. tocopherol, ascorbic acid), chelators (e.g. citric acid), organic or inorganic acidulants (e.g. malic acid, acetic acid, citric acid, tartaric acid, phosphoric acid), bitter substances (e.g. quinine, caffeine, limonin, amarogentin, humulones, lupulones, catechols, tannins), mineral salts (e.g. sodium chloride, potassium chloride, magnesium chloride, sodium phosphates), substances inhibiting enzymatic browning (e.g. sulfite, ascorbic acid), essential oils, plant extracts, natural or synthetic colorants or colour pigments (e.g. carotenoids, flavonoids, anthocyans, chlorophyll and derivatives thereof), spices, trigeminally active substances or plant extracts containing these trigerninally active substances, synthetic, natural or nature-identical flavourings or fragrances and odour-masking agents.

Dental care agents (as a base for preparations used for oral hygiene) containing 4-phenylpentan-2-ol generally include an abrasive system (abradants or polishing agents), such as e.g. silicas, calcium carbonates, calcium phosphates, aluminium oxides and/or hydroxyl apatites, surface-active substances, such as e.g. sodium lauryl sulfate, sodium lauryl sarcosinate and/or cocamidopropyl betaine, humectants, such as e.g. glycerol and/or sorbitol, thickeners, such as e.g. carboxymethylcellulose, polyethylene glycols, carrageenan and/or Laponite®, sweeteners, such as e.g. saccharin, flavour-masking agents for unpleasant flavour impressions, flavour-masking agents for other, generally not unpleasant, flavour impressions, flavour-modulating substances (e.g. inositol phosphate, nucleotides such as guanosine monophosphate, adenosine monophosphate or other substances such as monosodium glutamate or 2-phenoxypropionic acid), cooling substances, such as e.g. menthol, menthol derivatives (e.g. L-menthol, L-menthyl lactate, L-menthyl alkyl carbonates, menthone ketals, menthane carboxamides), 2,2,2-trialkylacetamides (e.g. 2,2-diisopropylpropionic acid methylamide), icilin and icilin derivatives, stabilisers and active substances, such as e.g. sodium fluoride, sodium monofluorophosphate, tin difluoride, quaternary ammonium fluorides, zinc citrate, zinc sulfate, tin pyrophosphate, tin dichloride, mixtures of different pyrophosphates, triclosan, cetylpyridinium chloride, aluminium lactate, potassium citrate, potassium nitrate, potassium chloride, strontium chloride, hydrogen peroxide, flavours and/or sodium bicarbonate or odour masking agents.

Chewing gums (as another example of preparations used for oral hygiene) containing 4-phenylpentan-2-ol generally comprise a chewing gum base, i.e. a masticatory substance becoming plastic during chewing, sugars of various types, sugar substitutes, other sweet-tasting substances, sugar alcohols, flavour-masking agents for unpleasant flavour impressions, other flavour modulators for other, generally not unpleasant, flavour impressions, flavour-modulating substances (e.g. inositol phosphate, nucleotides such as guanosine monophosphate, adenosine monophosphate or other substances such as monosodium glutamate or 2-phenoxypropionic acid), humectants, thickeners, emulsifiers, flavours and stabilisers or odour masking agents.

In addition to 4-phenylpentan-2-ol, the preparations according to the invention preferably contain a flavour composition to round out and refine the taste and/or odour of the preparation. Suitable (additional) flavour compositions contain e.g. synthetic, natural or nature-identical flavourings, fragrances and taste substances as well as suitable auxiliary substances and supports.

Other aspects of the present invention can be taken from the following examples and the attached claims.

The invention is explained in more detail below on the basis of examples. Unless otherwise stated, all data relate to the weight.

### EXAMPLE 1

### Preparation of 4-phenylpentan-2-ol formula (I) (Based on the Method According to the Literature Journal f. prakt. Chemie vol. 319, Part 4, 1977, 601-610)

206 g (1 mol) 2,4,6-trimethyl-4-phenyl-(1,3)-dioxane (Vertacetal®) in 250 ml ethanol/water 9:1 were initially charged into a 1 I stirred autoclave with 0.2 g palladium (5% on activated carbon). Hydrogenation was then performed at 1 80° C. under a pressure of 20 bar for 8 hours with stirring until 2 mol hydrogen were taken up. After cooling to room temperature, the reaction solution was then filtered and concentrated. The crude product (165 g) was fractionated in a 30 cm Vigreux column in vacuo.

Yield: 150.9 g (92% of theoretical value) Bp.: 130⁰-135° C./8 mbar

Purity according to GC 99.5%, diastereomer ratio (approx. 1:1)

### EXAMPLE 2

### Preparation of 4-phenylpentan-2-ol of Formula (I) via Grignard Reaction

148 g (1 mol) 3-phenylbutyraldehyde in 500 ml THF abs. were initially charged into a 2 l mixer. 81.8 g methyl magnesium chloride (22% solution in THF (1.1 mol)) were then added within one hour with stirring and cooling at 0° C. Stirring was then continued for 1.5 hours under reflux. After cooling to room temperature, the reaction solution was carefully separated with 300 ml 10% NaOH, the aqueous phase was separated off and the organic phase was washed with water until neutral and concentrated. The crude product (167.8 g) was fractionated in a 30 cm Vigreux column in vacuo.

Yield: 147 g (89.6% of theoretical value) Bp.: 130° -135° C/8 mbar.

Purity according to GC 99.5%, diastereomer ratio (approx. 1:1)

The spectroscopic data were determined for 4-phenylpentan-2-ol of formula (I). The data of the diastereomers (approx. 1:1) are given below.

¹H-NMR (CDCl₃, 400 MHZ, TMS=0): 1.12, 1.18 (d, J=6.2, 3H); 1.26, 1.27 (d, J=7, 3H); 1.61-1.86 (m,2H); 2.81-3.01 (m, 1H); 3.53-3.59 (m, 1H); 7.18-7.32 (m, aromat. 5H).

¹³C-NMR (CDCl₃, 100 MHz): 147.3 (s), 146.8 (s); 128.6 (d), 128.5 (d);127.1 (d), 126.9 (d); 126.2 (d), 126.1 (d);66.4 (d), 65.9 (d); 47.9 (t), 47.7 (t); 37.0 (d), 36.6 (d); 24.2 (q), 23.7 (q); 23.1 (q), 22.3 (q).

MS: m/z (%)=164 (3, M⁺), 146 (32), 131 (89), 106 (49), 105 (100), 91 (55), 78 (20), 77 (24), 45 (18).

MS: m/z (%)=164 (3, M⁺), 146 (34), 131 (98), 106 (45), 105 (100), 91 (55), 78 (20), 77 (25),45 (18).

EXAMPLE 3
Perfume composition (fragrance composition)

| | Parts by weight |
|---|---|
| Agrumex LC | 10.00 |
| Amarocit ® 10% in DPG | 10.00 |
| Ambroxide | 10.00 |
| Basil Oil | 10.00 |
| Calone 1951 10% in DPG | 10.00 |
| Cedarwood Oil | 10.00 |
| Cedrol | 50.00 |
| Citral 10% in DPG | 10.00 |
| Citronellol | 5.00 |
| Coumarin | 10.00 |
| Cyclogalbanat ® 10% in DPG | 15.00 |
| Dihydromyrcenol | 80.00 |
| Farenal ® 10% in DPG | 5.00 |
| Galbex 10% in DPG | 25.00 |
| Globalide ® | 80.00 |
| Globanone ® | 40.00 |
| Hedione | 90.00 |
| Helional | 20.00 |
| Heliotropin | 5.00 |
| Hexenol cis-3 10% in DPG | 15.00 |
| Hexenyl Salicylate cis-3 | 10.00 |
| beta-Ionone | 5.00 |
| Iso E Super | 180.00 |
| Isodamascon ® 10% in DPG | 10.00 |
| Isomuscone (Cyclohexadecanone) | 20.00 |
| Isoraldein 70 | 20.00 |
| Ketamber 10% in TEC | 25.00 |
| Lavandin Oil Grosso Nat. | 15.00 |
| Lilial | 20.00 |
| Linalool | 20.00 |
| Linalyl Acetate | 40.00 |
| Mandarin Oil Brazil, green | 50.00 |
| Timberol ® | 40.00 |
| Vanillin | 5.00 |
| Veloutone 10% in DPG | 20.00 |
| Ysamber K ® | 10.00 |
| Total | 1000.00 |

DPG: Dipropylene glycol,
TEC = Triethyl citrate

Odour description of the perfume composition without addition of the compound of formula (I): woody, floral, sharp

In the perfumers' opinion, by adding 3 wt. % 4-phenylpentan-2-ol from example 2 this perfume composition becomes fresher, more radiant, more rounded and more harmonious, with a green-fruity and sweet note being added and the woody and floral aspects being intensified. The 4-phenylpentan-2-ol of formula (I) used gives the composition a character of its own thanks to its inherent odour and its modifying and intensifying effect (booster effect) and binds the different odour elements together.

### EXAMPLE 4

### Perfume Composition (Fragrance Composition)

EXAMPLE 4
Perfume composition (fragrance composition)

| | Parts by weight |
|---|---|
| Allyl Cyclohexylpropionate | 3.00 |
| Amyl Salicylate | 2.00 |
| Benzyl Acetate | 64.00 |
| Citronellol | 122.00 |
| Citral 10% in DPG | 2.00 |
| Cyclamenaldehyde | 10.00 |
| Dihydromyrcenol | 3.00 |
| Dimethylbenzylcarbinyl Acetate | 3.00 |
| Ethyl Salicylate 10% in DPG | 2.00 |
| Eugenol | 3.00 |
| Indoflor 10% in DPG | 16.00 |
| Galaxolide 50% in DPG | 164.00 |
| Geraniol | 35.00 |
| Dihydromethyl Jasmonate | 6.00 |
| Heliotropin | 4.00 |
| Hexylcinnamaldehyde | 121.00 |
| Vertocitral | 4.00 |
| Hedione | 42.00 |
| Indole | 2.00 |
| Isobutyl Salicylate | 6.00 |
| Lavandin Oil Grosso Nat. | 6.00 |
| Acetyl Cedrene | 10.00 |
| Majantol | 190.00 |
| Linalool | 35.00 |
| Linalyl Acetate | 10.00 |
| Methyl Anthranilate 10% in DPG | 5.00 |
| Nerol | 10.00 |
| Orange Oil | 6.00 |
| Phantolide | 4.00 |
| Phenylacetaldehyde Dimethyl Acetal | 6.00 |
| Phenylethyl Alcohol | 75.00 |
| Florol | 6.00 |
| Sandalwood Oil | 3.00 |
| Sandranol | 16.00 |
| Trifernal | 2.00 |
| Tonalide | 2.00 |
| Total | 1000.00 |

DPG: Dipropylene glycol

Odour description of the perfume composition without addition of the compound of formula (I): floral, lily of the valley.

In the perfumers+ opinion, by adding 6 wt. % 4-phenylpentan-2-ol of formula (I) from example 1 this perfume composition awakes to new life. The floral impression is considerably intensified. The composition has a more radiant, rounded and harmonious effect, with a green-fruity and natural note being added. The 4-phenylpentan-2-ol of formula (I) used gives the composition a character of its own thanks to its inherent odour and its modifying and intensifying effect (booster effect) and binds the different odour elements together.

By the further addition of 0.1 wt. % Vertacetal® the aforementioned aspects are emphasised still further in a harmonious manner.

### EXAMPLE 5

### Shampoo

4-Phenylpentan-2-ol of formula (I) from example 1 was incorporated at a dose level of 0.5 wt. % into a shampoo base of the following composition:

| | |
|---|---|
| Sodium lauryl ether sulfate | 12% |
| (e.g. Texapon NSO, Cognis Deutschland GmbH) | |
| Cocamidopropyl betaine | 2% |
| (e.g. Dehyton K, Cognis Deutschland GmbH) | |
| Sodium chloride | 1.4% |
| Citric acid | 1.3% |
| Phenoxyethanol, methyl-, ethyl-, butyl- | 0.5% |
| and propylparaben | |
| Water | 82.8% |

The pH value of the shampoo base was about 6.100 ml of a 20 wt. % aqueous shampoo solution were produced from this. Two strands of hair were washed together in this shampoo solution for 2 minutes and then rinsed for 20 seconds under running, hand-hot water. One strand of hair was packed wet into aluminum foil and the second strand of hair was dried using a hairdryer. Both strands of hair were evaluated in terms of their odour by a panel.

Odour description in each case: radiant, interesting green perfume note in the direction of grapefruit and rhubarb, very natural radiance.

### EXAMPLE 6

### Fabric Softener

The perfume composition from example 3 (after adding 3 wt. % 4-phenylpentan-2-ol formula (1) from example 2) was incorporated at a dose level of 0.5 wt. % into a fabric softener base of the following composition:

| | |
|---|---|
| Quaternary ammonium methosulfate (Esterquat), approx. 90% | 5.5% |
| (e.g. Rewoquat WE 18, Witco Surfactants GrnbH) | |
| Alkyldimethylbenzylammonium chloride, approx. 50% | 0.2% |
| (e.g. Preventol R50, Bayer AG) | |
| Colour solution, approx. 1% | 0.3% |
| Water | 94.0% |

The pH value of the fabric softener base was in the range of 2 to 3. Two pieces of cloth were rinsed for 30 minutes at 20° C. in the final rinse programme of a line test machine with 370 g of a 1% aqueous fabric softener solution based on the fabric softener base containing 0.5% wt. % of compound formula (I). The cloths were wrung out and then spun for 20 seconds. One cloth was shrink-wrapped while wet and one was hung up to dry. The two cloths then had their odour evaluated by a panel.

Odour description in each case: floral, woody, fresh, radiant, green-fruity aspects with light sweet undertones, rounded and harmonious odour impression.

### EXAMPLE 7

### Washing Powder

The perfume composition from example 4 (after adding 6 wt. % of compound formuls (I) from example 1) was incorporated at a dose level of 0.4 wt. % into a washing powder base of the following formulation:

| | |
|---|---|
| Linear Na-alkylbenzene sulfonate | 8.8% |
| Ethoxylated fatty alcohol C12-18 (7 EO) | 4.7% |
| Na soap | 3.2% |
| Defoamer | |
| DOW CORNING(R) 2-4248S | |
| POWDERED ANTIFOAM, | |
| Silicone oil on zeolite as support material | 3.9% |
| Zeolite 4A | 28.3% |
| Na carbonate | 11.6% |
| Na salt of a copolymer of acrylic | 2.4% |
| and maleic acid (Sokalan CP5) | |
| Na silicate | 3.0% |
| Carboxymethylcellulose | 1.2% |
| Dequest 2066 | 2.8% |
| ([[(Phosphonomethyl)imino]bis[(ethylenenitrilo)bis | |
| (methylene)]]tetrakisphosphonic acid, sodium salt) | |
| Optical brightener | 0.2% |
| Na sulfate | 6.5% |
| Protease | 0.4% |
| Sodium perborate tetrahydrate | 22.0% |
| TAED | 1.0% |

Two pieces of cloth were washed for 45 minutes at 60° C. in the main wash programme of a line test machine with 370 g of a 1% aqueous washing powder solution based on the washing powder base containing 0.4 wt. % of the perfume oil composition from example 4 (the pH value of the washing powder solution is clearly within the basic range). The cloths were then rinsed with cold water for 5 minutes, wrung out and then spun for 20 seconds. One cloth was shrink-wrapped while wet and one was hung up to dry. The two cloths then had their odour evaluated by a panel.

Odour description in each case: strongly floral, radiant, green-fruity and natural note with light sweet and woody undertones, rounded and harmonious odour impression.

What is claimed is:

1. A fragrance or flavoring, comprising 4-phenylpentan-2-ol (I)

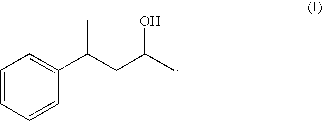

2. A fragrance or flavouring with a green, fruity note, comprising 4-phenylpentan-2-ol (I)

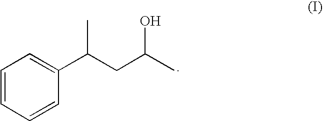

3. The method of imparting, modifying and/or intensifying an odour and/or flavour note with the following aspects: green, fruity, by administering the flavouring or fragrance of claim 1.

4. A perfumed or flavoured article containing
(a) 4-phenylpentan-2-ol (I) 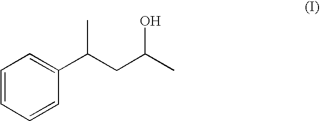
and
(b) one, two three or more other fragrances or flavourings.

5. The perfumed or flavoured article according to claim 4, wherein the other fragrance(s) or flavouring(s) (component (b)) impart(s) a woody and/or floral odour and/or flavour.

6. The perfumed or flavoured article according to claim 4, containing as another fragrance 2,4,6-trimethyl-4-phenyl-1,3-dioxane.

7. The perfumed or flavoured article according to claim 4, wherein the weight ratio of the total quantity of 4-phenylpentan-2-ol to the total quantity of other fragrances or flavourings is in the range of 1: 1000 to 1: 0.5.

8. The perfumed or flavoured article according to claim 4, wherein the article is a fragrance or flavouring composition.

9. The fragrance or flavouring composition according to claim 8, containing 4-phenylpentan-2-ol in a quantity sufficient to impart, to modify and/or to intensify an odour or flavour note of the green, fruity type.

10. The fragrance or flavouring composition according to claim 8, characterised in that the fragrance or flavouring composition contains a total quantity of 4-phenylpentan-2-ol in the range of 0.01 to 50 wt. %, based on the total quantity of the fragrance or flavouring composition.

11. The perfumed or flavoured article according to claim 4, wherein the article is selected from the group consisting of: perfume extracts, eaux de parfum, eaux de toilette, aftershaves, eaux de cologne, pre-shave products, splash colognes, perfumed freshening wipes, acidic, alkaline and neutral cleaners, textile fresheners, ironing aids, liquid laundry detergents, powdered laundry detergents, laundry pre-treatment agents, fabric softeners, laundry soaps, laundry tablets, disinfectants, surface disinfectants, air fresheners, aerosol sprays, waxes and polishes, toiletries, hand creams and lotions, foot creams and lotions, depilatory creams and lotions, aftershave creams and lotions, tanning creams and lotions, hair-care products, deodorants and antiperspirants, decorative cosmetic products, candles, lamp oils, incense sticks, insecticides, repellents and propellents.

12. A process for imparting, modifying and/or intensifying an odour or flavour with one or more or all of the notes green, fruity, wherein a sensorially active quantity of (i) 4-phenylpentan-2-ol of formula (I) 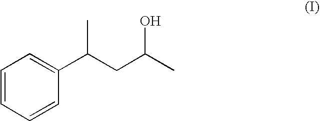
or
(ii) a fragrance or flavouring composition containing 4-phenylpentan-2-ol is brought into contact or mixed with a product.

13. A process for the preparation of (i) 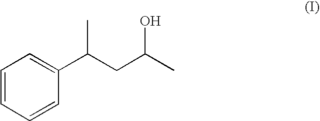
or (ii) of a mixture containing 4-phenylpentan-2-ol, with the following step:
catalytic cleavage of a substituted 1,3-dioxane of formula (A) 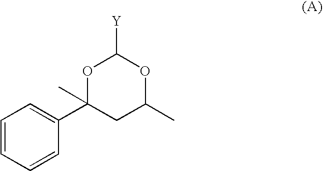
wherein Y signifies hydrogen or an acyl residue with 1 to 10 C atoms.

14. The process according to claim 13, wherein Y signifies methyl or ethyl.

15. The process according to claim 13, wherein Y signifies methyl.

16. The process according to claim 13, wherein the mixture (ii) containing 4-phenylpentan-2-ol contains a proportion of the substituted 1,3-dioxane of formula (A).

17. The fragrance or flavouring composition of claim 10, wherein the composition contains 0.01 to 25 wt % of 4-phenylpentan-2-ol based on the total weight of the composition.

18. The fragrance of flavouring composition of claim 10, wherein the composition contains 0.5 to 20 wt % of 4-phenylpentan-2-ol based on the total weight of the composition.
